(19) United States
(12) Patent Application Publication
(10) Pub. No.: US 2022/0054270 A1
(43) Pub. Date: Feb. 24, 2022

(54) MITRAL LEAFLET REPAIR

(71) Applicant: Edward Lifesciences Corporation, Irvine, CA (US)

(72) Inventors: Boaz Manash, Givat Ada (IL); Eitan Atias, Tel Aviv (IL); Marat Kveler, Nesher (IL); Adi Carmi, Ganei Tikva (IL)

(21) Appl. No.: 17/520,435

(22) Filed: Nov. 5, 2021

Related U.S. Application Data
(63) Continuation of application No. PCT/US2020/029218, filed on Apr. 22, 2020.
(60) Provisional application No. 62/845,004, filed on May 8, 2019.

(51) Int. Cl. A61F 2/24

(52) U.S. Cl. CPC A61F 2/2454; A61F 2/2466

(57) ABSTRACT

A method and device for placing a leaflet patch over a heart valve. The device comprises a first atrium anchor and a first ventricle anchor configured to be placed in an atrium, a second atrium anchor and a second ventricle anchor configured to be placed in a ventricle, a first artificial chord configured to attach to the first atrium anchor and the first ventricle anchor, a second artificial chord configured to attach to the second atrium anchor and the second atrium anchor, and the leaflet patch. The leaflet patch is placed partially in the atrium and partially in the ventricle. The leaflet patch comprises a first channel configured to contain the first artificial chord and a second channel configured to contain the second artificial chord. At least one of the anchors comprises a winching mechanism for adjusting the tension of one of the articular chords.

### RELATED APPLICATIONS

This application is a continuation of International Patent Application Serial No. PCT/US2020/029218, filed on Apr. 22, 2020 and entitled MITRAL LEAFLET REPAIR, which claims priority based on U.S. Provisional Patent Application Ser. No. 62/845,004, filed on May 8, 2019 and entitled MITRAL LEAFLET REPAIR, the full disclosures of both of which are hereby incorporated by reference in their entireties.

### BACKGROUND

The present disclosure generally relates to the field of heart valve correction. Heart valve dysfunction can result in regurgitation and other complications due to valve prolapse from failure of valve leaflets to properly coapt. For the mitral valve, the inability to close tightly can allow blood to flow backwards into the left atrium.

### SUMMARY

In some implementations, the present disclosure relates to a system and method for treating a heart valve. A leaflet patch can be implanted over the mitral valve to help restrain excessive leaflet motion. Restraining the leaflet motion can help fix the coaptation between the posterior and anterior leaflets of the mitral valve, potentially preventing or at least reducing the amount of blood flowing backwards into the left atrium

In some embodiments, artificial chords are anchored to papillary muscles and connected to a leaflet patch. In one embodiment, the leaflet patch comprises channels or tubes through which the artificial chords run. The leaflet patch can slide into place using the artificial chords. In one embodiment, the other ends of the artificial chords are anchored above the patch in the left atrium, for example, on the bottom of the left atrium on the posterior side. In some embodiments, winching mechanism built into the left atrium anchors (or in the ventricle anchors) allow artificial chords to be wound tighter, generating greater tension by the leaflet patch on the mitral valve. The artificial chords can be tightened until the excessive leaflet motion of the mitral valve is sufficiently restrained to eliminate or at least reduce mitral regurgitation.

In some implementations, the present disclosure relates to a device for placing a leaflet patch over a heart valve. The device comprises a first atrium anchor configured to be placed in an atrium of a heart, a second atrium anchor configured to be placed in the atrium, a first ventricle anchor configured to be placed in a ventricle of the heart, a second ventricle anchor configured to be placed in the ventricle, a first artificial chord configured to attach to the first atrium anchor and the first ventricle anchor, a second artificial chord configured to attach to the second atrium anchor and the second atrium anchor, and a leaflet patch configured to cover a leaflet of a heart valve, the leaflet patch partially in the atrium and partially in the ventricle. The leaflet patch comprises a first channel configured to contain the first artificial chord and a second channel configured to contain the second artificial chord.

In some embodiments, the first atrium anchor comprises an anchor base attached to a tissue anchor, the anchor base configured to rotate with a catheter and a winch drum configured to rotate with an inner shaft, and rotation of the winch drum is independent from rotation of the anchor base and the rotation of the winch drum causes the first artificial chord to wind around the winch drum. For example, a key of the inner shaft may be configured to engage with a slot of the winch drum in a first position of the inner shaft proximal to a tissue site. In some embodiments, the second atrium anchor comprises a second anchor base attached to a second tissue anchor, the second anchor base configured to rotate with a second catheter and a second winch drum configured to rotate with a second inner shaft, and rotation of the second winch drum is independent from rotation of the second anchor base and the rotation of the second winch drum causes the second artificial chord to wind around the second winch drum. In some embodiments, the catheter is configured to rotatably engage with the anchor base and rotation of the catheter is configured to drive the anchor base into a tissue site. In some embodiments, the inner shaft is configured to rotatably engage with a slot of the winch drum, rotation of the inner shaft is configured to rotate the winch drum, the inner shaft is located within the catheter, and the inner shaft and the catheter are independently rotatable. For example, the catheter comprising the inner shaft can be pre-attached to the first atrium anchor.

The device may further comprise a transseptal guide sheath for inserting the device into the heart. In some embodiments, the heart valve is a mitral valve and the leaflet patch is configured to cover a posterior leaflet of the mitral valve. In some embodiments, the first channel is formed on a first edge of the leaflet patch and the second channel is formed on a second edge of the leaflet patch. The leaflet patch may be substantially rectangular. In some embodiments, the first artificial chord is pre-attached to the first atrium anchor and the first ventricle anchor. For example, the second artificial chord can be pre-attached to the second atrium anchor and the second ventricle anchor.

In some implementations, the present disclosure relates to a method for treating a heart valve. The method comprises attaching a first ventricle anchor in a ventricle of a heart, the first ventricle anchor being connected to a first artificial chord, and attaching a second ventricle anchor in the ventricle, the second ventricle anchor connected to a second artificial chord. The method further comprises placing a leaflet patch across a heart valve, the leaflet patch partially in an atrium of the heart and partially in the ventricle, the leaflet patch comprising a first channel and a second channel, the first artificial chord running through first channel, the second artificial chord running through the second channel. The method further comprises attaching a first atrium anchor in the atrium, the first atrium anchor connected to the first artificial chord and attaching a second atrium anchor in the atrium, the first atrium anchor connected to the first artificial chord.

The method may further comprise adjusting the first artificial chord by using a first winching mechanism of the first atrium anchor. In some embodiments, the method further comprises adjusting the second artificial chord by using a second winching mechanism of the second atrium anchor. In some embodiments, the method further comprises monitoring mitral valve regurgitation during the adjustment of the first artificial chord, wherein the adjustment is based at least partly on the monitoring of the mitral valve regurgitation. In some embodiments, attaching the first atrium anchor comprises driving the first atrium anchor into a tissue site of the heart using a catheter rotatably engaged with the first atrium anchor. For example, the method may further comprise holding the catheter stationary and adjusting the first artificial chord by independently rotating an inner shaft within the catheter, the inner shaft rotatably engaged with a winching mechanism of the first atrium anchor.

In some implementations, the present disclosure relates to a device for placing a leaflet patch over a heart valve. The device comprises a first artificial chord configured to attach to a first anchor in a first chamber of the heart and a second anchor in a second chamber of the heart, a second artificial chord comprising a first end configured to attach to a third anchor in the second chamber of the heart and a second end configured to attached to the first anchor or a fourth anchor in the first chamber of the heart, and a leaflet patch configured to cover a leaflet of a heart valve, the leaflet patch partially in the first chamber and partially in the second chamber. The leaflet patch comprises a first channel configured to contain the first artificial chord and a second channel configured to contain the second artificial chord.

### BRIEF DESCRIPTION OF THE DRAWINGS

Various embodiments are depicted in the accompanying drawings for illustrative purposes, and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 provides a cross-sectional view of a human heart.

FIG. 2 provides a cross-sectional view of the left ventricle and left atrium of an example heart.

FIG. 3 provides a cross-sectional view of a heart experiencing mitral regurgitation.

FIG. 4 illustrates a profile perspective of a leaflet patch device placed in a portion of the heart shown in FIG. 3, in accordance with some embodiments.

FIG. 5 illustrates a facing perspective the leaflet patch device, in accordance with some embodiments.

FIGS. 6A-6C illustrate various stages during an implantation of the leaflet patch device, according to certain embodiments.

FIGS. 7A and 7B illustrate anchors of the leaflet patch device attached to a leaflet patch using artificial chords, in accordance with some embodiments.

FIG. 8 illustrates a perspective view of the leaflet patch device showing catheters for engaging with the anchors of the leaflet patch device, in accordance with some embodiments

FIG. 9 illustrates a perspective view of the leaflet patch device after implantation is completed, in accordance with some embodiments.

FIG. 10 illustrates a side view of catheter of the leaflet patch device, in accordance with some embodiments.

FIG. 11 illustrates a cross-sectional view of the catheter of FIG. 10, according to some embodiments.

FIG. 12 illustrates the catheter of FIG. 10 with an external section cut-out, according to some embodiments.

FIG. 13 illustrates a perspective view of a winch drum of the left atrium anchor, in accordance with some embodiments.

FIG. 14 provides a flow diagram representing a process for implanting a leaflet patch device, according to some embodiments.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, with respect to any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or apparatuses/devices described herein may be embodied as integrated components or as separate components. For purposes of comparing various embodiments, certain aspects and advantages of these embodiments are described.

The headings provided herein are for convenience only and do not necessarily affect the scope or meaning of the claimed invention.

Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein. Similar reference numbers may be used with respect to separate diagrams and/or embodiments; use of such similar, or identical, reference numbers should not be interpreted as necessarily identifying identical components, and may refer to separate features. Overview

Mitral regurgitation, mitral insufficiency or mitral incompetence—is a condition in which a heart's mitral valve does not close tightly, allowing blood to flow backward into the left atrium. If the mitral valve regurgitation is significant, blood cannot move through the heart or to the rest of your body as efficiently, making a person feel tired or out of breath. Left untreated, severe mitral valve regurgitation can cause heart failure or heart rhythm problems (arrhythmias).

The most common leaflet dysfunction in degenerative valve disease is Type II, excess motion of the margin of the leaflet in relation to the annular plane. The lesions in degenerative valve disease that result in the Type II dysfunction are usually chordae elongation or rupture. Annular dilatation is almost always an associated finding.

Type I dysfunction with normal leaflet motion and pure annular dilatation is a less common form of degenerative valve disease. It may be associated with conditions that result in significant atrial dilatation such as long-standing atrial fibrillation, or may occur in patients with connective tissue disorders.

The following disclosure discuses a leaflet patch device that can be used to treat mitral regurgitation that occurs due to chord tears and/or malcoaptation of anterior and posterior leaflets. In some embodiments, the device can treat type 2A and 2B mitral regurgitation with increased leaflet motion and can also treat functional mitral regurgitation.

### Implantation Location

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.).

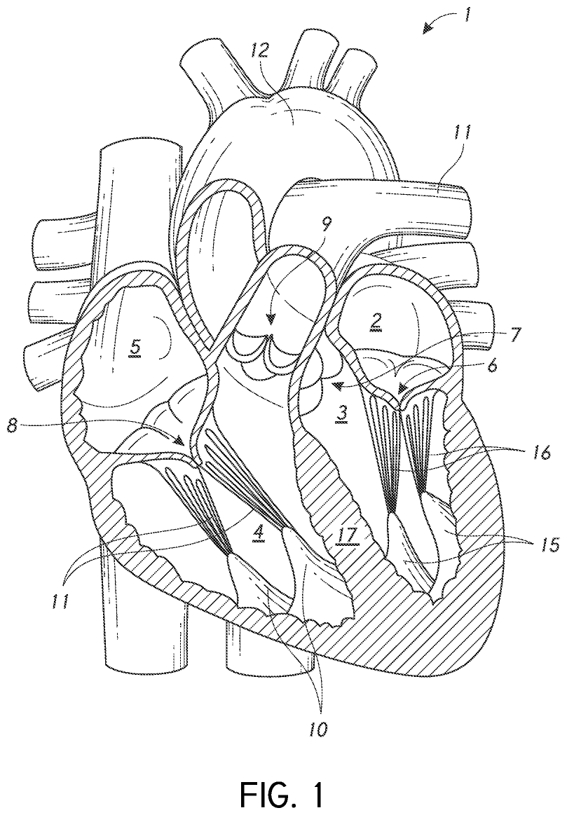

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 11, and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size and position of the leaflets or cusps may be such that when the heart contracts, the resulting increased blood pressure produced within the corresponding heart chamber forces the leaflets at least partially open to allow flow from the heart chamber. As the pressure in the heart chamber subsides, the pressure in the subsequent chamber or blood vessel may become dominant, and press back against the leaflets. As a result, the leaflets/cusps come in apposition to each other such that the leaflets/cusps coapt, thereby closing the flow passage.

The atrioventricular (i.e., mitral 6 and tricuspid 8) heart valves may further comprise a respective collection of chordae tendineae (16, 11) and papillary muscles (15, 10) for securing the leaflets of the respective valves to promote and/or facilitate proper coaptation of the valve leaflets and prevent prolapse thereof. The papillary muscles (15, 10) may generally comprise finger-like projections from the ventricle wall, while the chordae tendineae (16, 10) may comprise cord-like tendons that connect the papillary muscles to the valve leaflets.

With respect to the mitral valve 6, a normal mitral valve may comprise two leaflets (anterior and posterior) and chordae tendineae 16 connecting the leaflets to two corresponding papillary muscles 15. The papillary muscles 15 originate in the left ventricle wall and project into the left ventricle 3. The valve leaflets of the mitral valve 6 may be prevented from prolapsing into the left atrium 2 by the action of the chordae tendineae 16 tendons connecting the valve leaflets to the papillary muscles 15. The relatively inelastic chordae tendineae 16 are attached at one end to the papillary muscles 15 and at the other to the valve leaflets; chordae tendineae from each of the papillary muscles 15 are attached to a respective leaflet of the mitral valve 6. Thus, when the left ventricle 3 contracts, the intraventricular pressure can force the valve to close, while the chordae tendineae 16 may keep the leaflets coapting together and prevent the valve from opening in the wrong direction, thereby preventing blood to flow back to the left atrium 2. The various cords of the chordae tendineae may have different thicknesses, wherein relatively thinner cords are attached to the free leaflet margin, while relatively thicker cords (e.g., strut cords) are attached farther away from the free margin.

With respect to the tricuspid valve 8, a normal tricuspid valve may comprise three leaflets (two shown in FIG. 1) and three corresponding papillary muscles 10 (two shown in FIG. 1). The leaflets of the tricuspid valve 8 may be referred to as the anterior, posterior and septal leaflets, respectively. The valve leaflets are connected to the papillary muscles by the chordae tendineae 11, which are disposed in the right ventricle 4 along with the papillary muscles 10. Although tricuspid valves are described herein as comprising three leaflets, it should be understood that tricuspid valves may occur with two or four leaflets in certain patients and/or conditions; the principles relating to papillary muscle binding and/or adjustment disclosed herein are applicable to atrioventricular valves having any number of leaflets and/or chordae tendineae or papillary muscles associated therewith. The right ventricular papillary muscles 10 originate in the right ventricle wall, and attach to the anterior, posterior and septal leaflets of the tricuspid valve, respectively, via the chordae tendineae 11. The papillary muscles 10 may serve to secure the leaflets of the tricuspid valve 8 to prevent prolapsing of the leaflets into the right atrium 5 during ventricular systole. Tricuspid regurgitation can be the result of papillary dysfunction or chordae rupture.

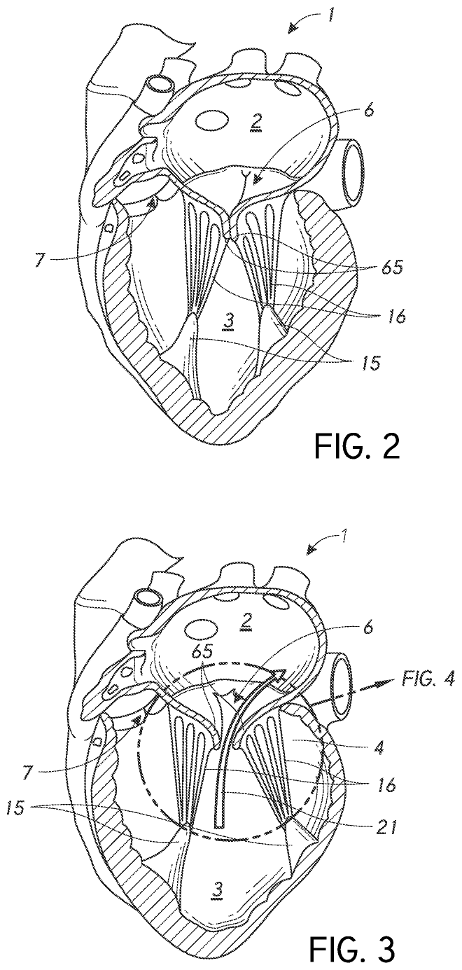

FIG. 2 provides a cross-sectional view of the left ventricle 3 and left atrium 2 of an example heart 1. The diagram of FIG. 2 shows the mitral valve 6, wherein the disposition of the valve 6, papillary muscles 15 and/or chordae tendineae 16 may be illustrative as providing for proper co-apting of the valve leaflets 65 to advantageously at least partially prevent regurgitation and/or undesirable flow into the left atrium from the left ventricle 3 and vice versa. Although a mitral valve 6 is shown in FIG. 2 and various other figures provided herewith, and described herein in the context of certain embodiments of the present disclosure, it should be understood that the leaflet patch device disclosed herein may be applicable with respect to any atrioventricular valve and associated anatomy (e.g., chordae tendineae, papillary muscles, ventricle wall, etc.), such as the tricuspid valve.

As described above, with respect to a healthy heart valve as shown in FIG. 2, the valve leaflets 65 may extend inward from the valve annulus and come together in the flow orifice to permit flow in the outflow direction (e.g., the downward direction in FIG. 2) and prevent backflow or regurgitation toward the inflow direction (e.g., the upward direction in FIG. 2). For example, during atrial systole, blood flows from the atria 2 to the ventricle 3 down the pressure gradient, resulting in the chordae tendineae 16 being relaxed due to the atrioventricular valve 6 being forced open. When the ventricle 3 contracts during ventricular systole, the increased blood pressures in both chambers may push the valve 6 closed, preventing backflow of blood into the atria 2. Due to the lower blood pressure in the atria compared to the ventricles, the valve leaflets may tend to be drawn toward the atria. The chordae tendineae 16 can serve to tether the leaflets and hold them in a closed position when they become tense during ventricular systole. The papillary muscles 15 provide structures in the ventricles for securing the chordae tendineae 16 and therefore allowing the chordae tendineae 16 to hold the leaflets 65 in a closed position. The papillary muscles 15 may include an anterolateral papillary muscle, which may be tethered to the posterior leaflet, for example, and a posteromedial papillary muscle, which may be tethered to the anterior leaflet, for example. With respect to the state of the heart 1 shown in FIG. 2, the proper coaptation of the valve leaflets, which may be due in part to proper position and/or tension of the chordae tendineae 16, may advantageously result in mitral valve operation substantially free of leakage.

Heart valve disease represents a condition in which one or more of the valves of the heart fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is in a closed state. In certain conditions, valve disease can be severely debilitating and even fatal if left insufficiently treated. With regard to incompetent heart valves, over time and/or due to various physiological conditions, the position and/or tension of the chordae tendineae and/or papillary muscles may become altered, thereby pulling the valve leaflets at least partly open, which may cause valve regurgitation. For example, functional mitral valve regurgitation can occur when the left ventricle of the heart is distorted or dilated, displacing the papillary muscles, and chordae tendineae attached thereto, that support the mitral valve leaflets. For example, the valve leaflets may no longer come together to close the annulus, thereby resulting in blood flow back into the atrium. If left untreated, functional mitral valve regurgitation can overload the heart and can lead to or accelerate heart failure. Moving or pulling the chordae tendineae closer to the flow axis of the valve annulus according to their natural and healthy positions can potentially reduce occurrence of valve regurgitation.

FIG. 3 illustrates a cross-sectional view of a heart 1 experiencing functional mitral valve regurgitation flow 21, which may be caused at least in part by dilation of the left ventricle. Left ventricle dilation can cause changes in the position of the papillary muscles 15, thereby allowing the flow 21 back from the ventricle 3 to the atrium 2. Dilation of the left ventricle can be causes by any number of conditions, such as focal myocardial infarction, global ischemia of the myocardial tissue, or idiopathic dilated cardiomyopathy, resulting in alterations in the geometric relationship between papillary muscles and other components associated with the valve(s) that can cause valve regurgitation. Functional regurgitation may further be present even where the valve components may be normal pathologically, yet may be unable to function properly due to changes in the surrounding environment. Examples of such changes include geometric alterations of one or more heart chambers and/or decreases in myocardial contractility. In any case, the resultant volume overload that may exist as a result of an insufficient valve may increase chamber wall stress, eventually resulting in a dilatory effect that causes papillary muscle and/or chordae tendineae alteration resulting in valve dysfunction and degraded cardiac efficiency.

When the chordae tendineae 16 are pulled away from the flow axis of the valve 6, the attached valve leaflets 65 may no longer come together sufficiently to close the annulus and prevent blood flow back into the atrium 2. Solutions presented herein provide devices and methods for controlling excess leaflet motion, which may advantageously reduce the occurrence of mitral regurgitation and/or improve coaptation. As shown in FIG. 3, the failure of the leaflets 65 of the mitral valve (or tricuspid valve) to come into a state of coaptation can result in an opening between the mitral valve leaflets 65 during the systolic phase of the cardiac cycle, which allows the leakage flow 21 of fluid back up into the atrium 2. In addition to the unwanted flow 21 in the outflow direction (e.g., the upward direction in FIG. 3), the failure of the valve leaflets 65 to coapt properly may result in unwanted backflow or regurgitation toward the inflow direction (e.g., the downward direction in FIG. 3) as well in certain conditions.

Various techniques that suffer from certain drawbacks may be implemented for treating mitral valve dysfunction, including surgical repair or replacement of the diseased valve or medical management of the patient, which may be appropriate/effective primarily in early stages of valve dysfunction, during which levels of regurgitation may be relatively low. For example, such medical management may generally focus on volume reductions, such as diuresis or afterload reducers, such as vasodilators, for example. Valve replacement operations may also be used to treat regurgitation from valve dysfunction. However, such operations can result in ventricular dysfunction or failure following surgery. Further limitations to valve replacement solutions may include the potential need for prolonged or lifelong therapy with powerful anticoagulants in order to mitigate the thromboembolic potential of prosthetic valve implants. Moreover, in the case of biologically-derived devices, such as those used as mitral valve replacements, the long-term durability may be limited. Another commonly employed repair technique involves the use of annuloplasty rings to improve mitral valve function. An annuloplasty ring may be placed in the valve annulus and the tissue of the annulus sewn or otherwise secured to the ring. Annuloplasty rings can provide a reduction in the annular circumference and/or an increase in the leaflet coaptation area. However, annuloplasty rings may flatten the saddle-like shape of the valve and/or hinder the natural contraction of the valve annulus. In addition, various surgical techniques may be used to treat valve dysfunction. However, such techniques may suffer from various limitations, such as requiring opening the heart to gain direct access to the valve and the valve annulus. Therefore, cardiopulmonary bypass may be required, which may introduce increased morbidity and mortality to the surgical procedures. Additionally, for surgical procedures, it can be difficult or impossible to evaluate the efficacy of the repair prior to the conclusion of the operation.

Various solutions disclosed herein relate to devices and methods for treating valve dysfunction without the need for cardiopulmonary bypass. Further, various embodiments disclosed herein provide for the treatment of valve dysfunction that can be executed on a beating heart, thereby allowing for the ability to assess the efficacy of the leaflet patch treatment and potentially implement modification thereto without the need for bypass support. Certain embodiments disclosed herein provide solutions for incompetent heart valves that involve applying a leaflet patch over the mitral valve to improve the seal formed by the leaflets of the mitral valve and prevent or reduce backward flow of blood into the left atrium.

### Leaflet Patch Device

As referenced above, certain embodiments disclosed herein provide for systems, devices and methods for applying a leaflet patch over the mitral valve (or other valve) of a heart in order to improve valve coaptation and prevent or reduce mitral regurgitation. Such devices may be introduced into the patient system through surgical or, advantageously, minimally-invasive means.

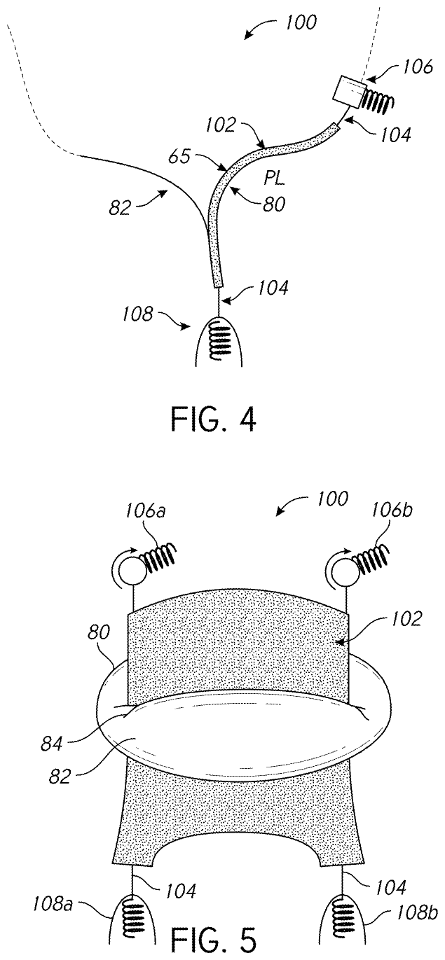

FIG. 4 illustrates a profile perspective of a leaflet patch device 100 placed in a portion of the heart 1 shown in the dashed circular area of FIG. 3 and described above, in accordance with some embodiments. FIG. 5 illustrates a facing perspective of the leaflet patch device 100 of FIG. 4.

In particular, FIGS. 4 and 5 show posterior leaflet 80 and anterior leaflet 82 (shown as valve leaflets 65 in FIGS. 1-3) in a closed state, with a coaptation area 84 formed by the contact of the posterior leaflet 80 and the anterior leaflet 82. In some embodiments, the leaflet patch device 100 comprises a leaflet patch 102, artificial chords 104, left atrium anchors 106, and left ventricle anchors 108. In some embodiments, the leaflet patch 102 is made out of pericardium, polytetrafluoroethylene (PTFE), expanded PTFE (ePTFE), other polymers, and/or other suitable material for implantation in the human body. In some embodiments, the artificial chords are made of ultra-high molecular weight polyethylene (e.g., Dyneema®), polymers, polyester, natural fibers, and/or other suitable material for implantation in the human body.

The artificial chords 104 can apply tension to the leaflet patch 102 to hold the leaflet patch 102 against the posterior leaflet 80. In some embodiments, engagement surfaces 109a, 109b are formed along the edges of the leaflet patch 102. The engagement surfaces can connect the leaflet patch to the artificial chord that, in one embodiment, run along the edges of the leaflet patch. In one embodiment, the engagement surfaces comprise tunnels or rails formed by the leaflet patch (e.g., edges of the patch folder over and connected by adhesive, suturing, or the like). In some embodiments, the engagement surfaces may also comprise adhesive, sutures, holes through which the artificial chord are inserted through, and/or other chemical or mechanical attachment mechanisms. By placing the artificial chords on the edges of the leaflet patch, the artificial chords can keep the leaflet patch under tension, potentially providing better contact between the leaflet patch and the posterior leaflet.

The artificial chord 104 can be anchored in the left ventricle by two left ventricle anchors 108a, 108b (shown in FIG. 5) and two left atrium anchors 106a, 106b (shown in FIG. 5). In some embodiments, the left atrium anchors include winches for adjusting the tension of the artificial chords 104. By allowing adjustments, the leaflet patch device 100 can better conform to the mitral valve. In one scenario, the echo measurements may be taken while the artificial chords are adjusted, in order to view how the tightening or loosening the artificial chords impacts mitral regurgitation. This can allow fine-tuning of the leaflet patch device 100 placement to potentially reach optimal or near optimal results in reducing or eliminating mitral regurgitation.

Each left atrium anchor 106a, 106b (collectively 106) may comprise a tissue anchor for attaching the left atrium anchors to the left atrium. The anchors may be placed into left atrium myocardium. In some embodiments, the left atrium anchors are pre-attached to the artificial chords 104, to simplify implantation.

Each left ventricle anchor 108a, 108b (collectively 108) may comprise a tissue anchor for attaching the left ventricle anchors to the left ventricle. The anchors may be placed into the left ventricle myocardium. In some embodiments, the left ventricle anchors are pre-attached to the artificial chords 104, to simplify implantation.

The atrium anchors 106, ventricle anchors 108, and/or tissue anchors may comprise of metal (e.g., nitinol, nickel, titanium, stainless steel, cobalt chrome, etc.), other rigid materials, and/or or other suitable implantable material. In one embodiment, the tissue anchors are formed into a helical coil to facilitate screwing the anchors into tissue. In other embodiments, the tissue anchors are screw-shaped, hook-shaped, anchor-shaped, or another configuration designed to attach to the tissue site.

While the above has discussed having a total of four anchors (two in the left atrium, two in the left ventricle), other embodiments of the leaflet patch device 100 can have different configurations of anchors. For example, there could be three total anchors in a 2-1 configuration, with two anchors on one side of the mitral valve (e.g., left ventricle or left atrium) and one anchor on the other side of the mitral valve. Another configuration could use five anchors in a 3-2 configuration, with three anchors on one side of the mitral valve (e.g., left ventricle or left atrium) and two anchors on the other side of the mitral valve. As will be apparent, other configurations with more than five anchors could also be used to potentially ensure less movement or greater rigidity in the leaflet patch device 100.

Other variations may also be possible. For example, the configuration of the left ventricle anchors 108 and the left atrium anchors 106 may be reversed, with the winching mechanism built into the left ventricle anchors 108 and/or the left atrium anchors pre-attached to the artificial chords 104.

Furthermore, while the above has described the anchors in terms of “left ventricle” or “left atrium” these terms are used to merely denote the location of the anchors in the specific scenario being described involving the mitral valve. The same anchors can be used in other locations of the heart, such as the right atrium or the right ventricle. For example, if the leaflet patch device 100 is used over a tricuspid valve, the anchors would be placed in the right atrium and the right ventricle.

The device 100 may comprise any suitable or desirable form, shape, or configuration, and may comprise any number of parts or components. For example, disclosed herein are embodiments of the device 100 comprising one or more anchors, catheters, chords, or the like. Procedures and methods for implanting the leaflet patch device in accordance with the present disclosure may involve transcatheter delivery, such as transfemoral, transseptal, transapical, or other type of transcatheter procedure.

### Leaflet Patch Implantation

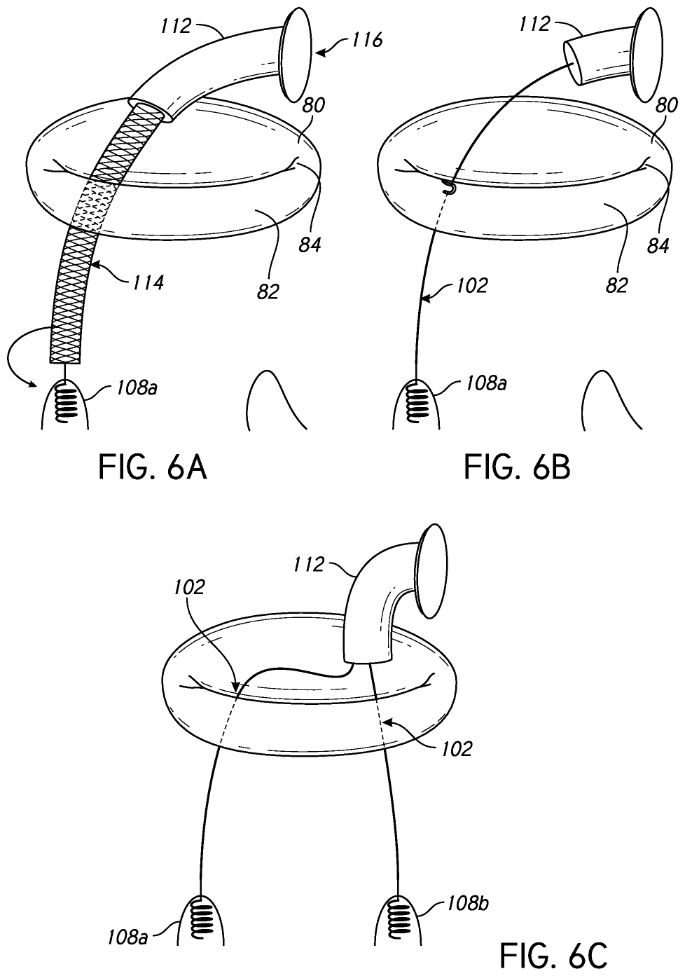

FIGS. 6A-6C illustrate various stages in the implantation of the leaflet patch device 100, according to certain embodiments. FIG. 6A illustrates one embodiment of a guide sheath 112 for introducing a torque shaft 114 into the left atrium. The guide sheath can be inserted through a fossa 116 or opening into the left atrium. In other implementations, the guide sheath may enter through other chambers of the heart, such as the left ventricle (for a mitral valve) or the right ventricle/atrium (for a tricuspid valve).

In one embodiment, the guide sheath 112 is inserted using a transseptal approach. In one implementation of the transseptal approach, the right atrium is opened and a longitudinal incision (e.g., about 4 cm) is made in the middle of the foramen ovalis on the intra-atrial septum. The septal edges are then pulled in order to expose the mitral valve fully. Other variations on the transseptal approach can change the point of incision in the septum. Ideally, such an incision provides access to the mitral valve annulus and the subvalvular apparatus, preferably exposing both completely and at a short surgical distance, thereby minimizing the chance of damage to surrounding structures. Other surgical approaches are also possible.

Once access to the mitral valve is gained, the guide sheath can be inserted from the left atrium, through the leaflets 80, 82 and into the left ventricle. A first left ventricle anchor 108a is then attached to the left ventricle myocardium. As discussed above, the left ventricle anchor 108a may be pre-attached to an artificial chord 104. In one embodiment, the left ventricle anchor is attached using a rotational anchoring method. For example, the torque shaft 114 may be configured to rotationally engage with left ventricle anchor such that a tissue anchor of the left ventricle anchor is driven or screwed into the myocardium tissue using a rotation motion of the torque shaft.

FIG. 6B illustrates the guide sheath 112 after the first left ventricle anchor 108a is inserted. The torque shaft 114 can be pulled out of the guide sheath 112, leaving the artificial chord 104 and left ventricle anchor 108a in place, attached in the left ventricle. The guide sheath 112 can then be moved to a second location, for better placement in installing the second left ventricle anchor 108b. At the second location, the torque shaft 114 is inserted into the guide sheath 112, through the mitral valve leaflets 80, 82 and into the left ventricle. The torque shaft 114 can be the same as the torque shaft used to install the first left ventricle anchor 108a or can be a second torque shaft. For example, the second torque shaft 114, the second left ventricle anchor 108b and the second artificial chord 104 may be prepared as a set for ease of insertion. Using the torque shaft 114, the second left ventricle anchor 108b is attached in the left ventricle.

FIG. 6C illustrates the guide sheath 112 after the left ventricle anchors 108a, 108b are installed in the left ventricle. The artificial chords 104 are also in place, ready for the leaflet patch 102 to be inserted over the mitral valve.

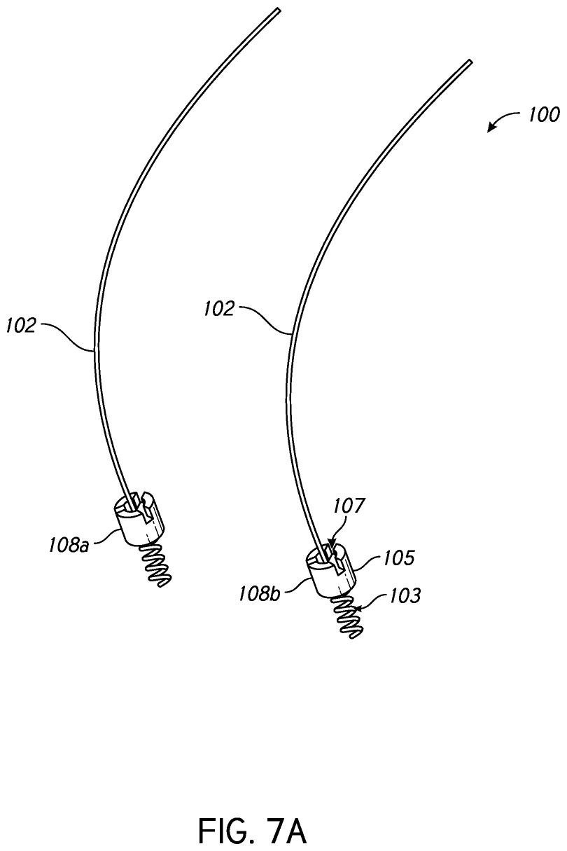

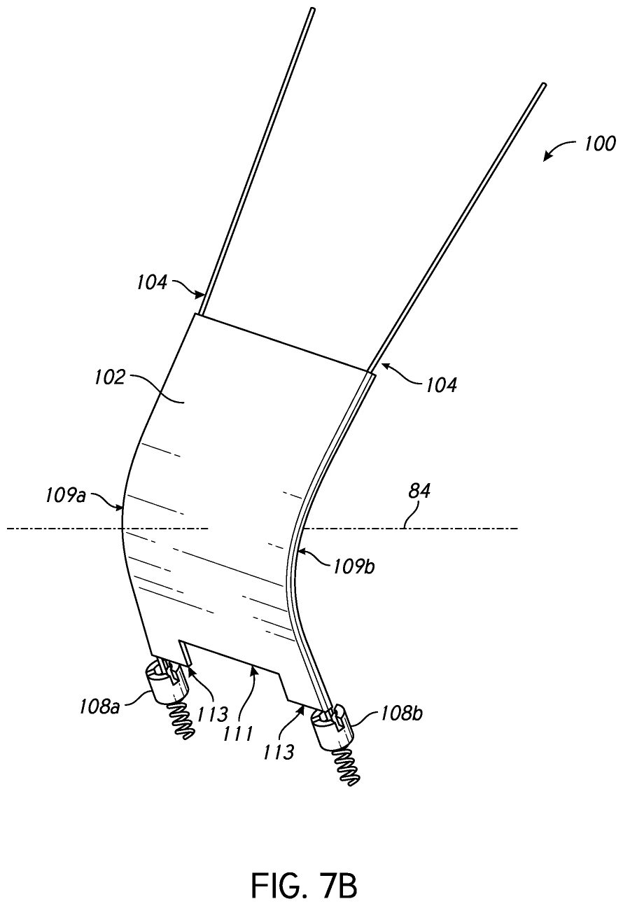

FIG. 7A illustrates two anchors 108a, 108b of the leaflet patch device 100, with artificial chords 104a, 104b connected to the anchors, in accordance with some embodiments. The left ventricle anchors 108a, 108b can be connected to the myocardium of the left ventricle. In one embodiment, the left ventricle anchors are connected to papillary muscles (15, 10) of the left ventricle.

As discussed above, the left ventricle anchors 108a, 108b can be made in a variety of forms. In the embodiment of FIG. 7A, each left ventricle anchor 108 comprises a tissue anchor 103 and an anchor base 105. The anchor base 105 can incorporates a drive 107 to facilitate rotational movement of the left ventricle anchors. In one embodiment, as shown, the drive comprises a tri-point, with three channels radiating from a central recess. The torque shaft 114 may have a proximal end to the left ventricle anchors configured engage with drive (e.g., shaped to fit into the three channels). As will be apparent, other types of drives can be used. For example, drive designs used in screws, such as slot, cross, Philips, hex, square triangle, torx, three-pointed, polydrive, bristol, ratchet-based or the like could be incorporated in the anchor base 105.

FIG. 7B illustrates a leaflet patch 102 attached to the artificial chords 104, in accordance with some embodiments. After the left ventricle anchors 108 and artificial chords 104 are placed in the left ventricle, the leaflet patch 102 can be installed using the artificial chords 104. In some embodiments, the leaflet patch comprises engagement surfaces 109a, 109b formed along the edges of the leaflet patch. For example, the engagement surfaces 109a, 109b may be rails or tunnels for the artificial chords 104, allowing the leaflet patch to slide onto the artificial cords during implantation.

In some embodiments, the leaflet patch 102 is substantially rectangular. A notch 111 may be formed on an edge of leaflet patch closest to the left ventricle anchors 108. The notch can define the inner boundary forming a pair of legs 113 that can engage with the anchor base 105. Other embodiments may utilize different shapes for the leaflet patch, such as an oval, triangle, square, diamond, pentagon, or the like. Shapes with more sides (e.g., 5 or more) may require additional channels in the leaflet patch as well as more corresponding anchors and artificial chords.

In one embodiment, a portion of the leaflet patch 102 is located in the left ventricle, below the leaflet coaptation area 84, as indicated by the dashed line. The remaining portion of the leaflet patch 102 lies above the coaptation area 84 in the left atrium.

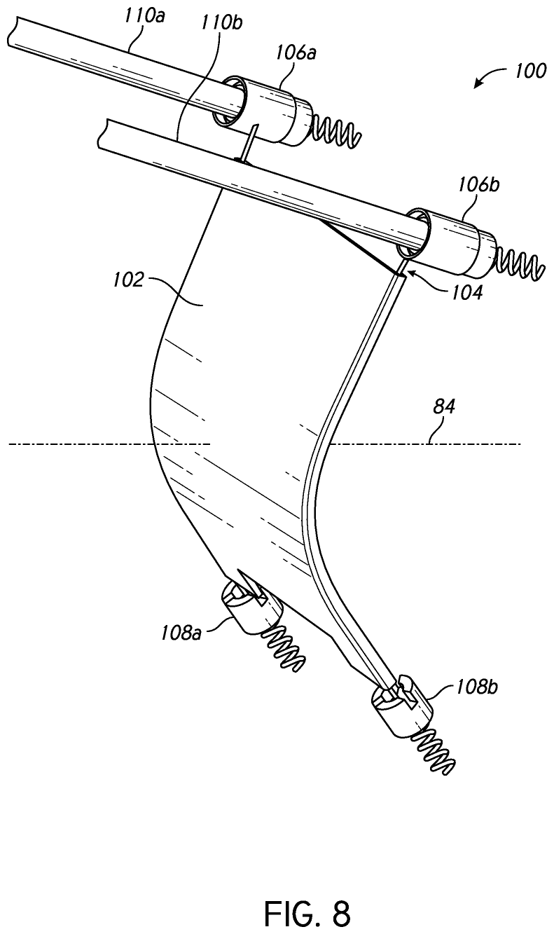

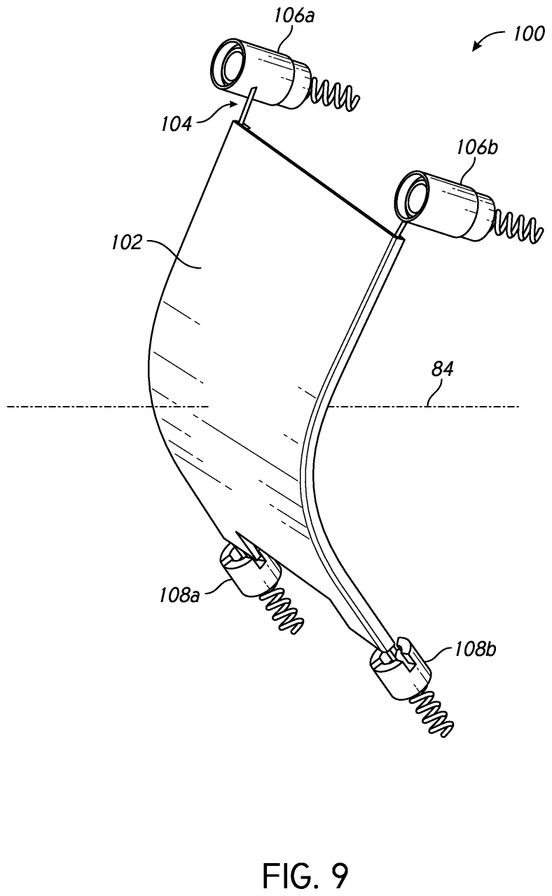

FIG. 8 illustrate catheters 110a, 110b (collectively 110) engaged with left atrium anchors 106 of the leaflet patch device 100, according to some embodiments. In the illustrated embodiment, a first catheter 110a is engaged with a first left atrium anchor 106a, while a second catheter 110b is engaged with a second left atrium anchor 106b. In other embodiments, a single catheter may be used to engage both left atrium anchors 106. For example, the single catheter may be used to drive the first left atrium anchor 106a and then be used to drive the second left atrium anchor 106b.

The catheters 110a, 110b can serve multiple functions. In one embodiment, the catheters, in a first configuration, are configured to drive the left atrium anchors 106 into the left atrium myocardium. For example, a rotational movement can be used to screw tissue anchors of the left atrium anchors 106 into a tissue site. In the same embodiment, the catheters are configured to engage a winching mechanism of the left atrium anchors 106 in order to tighten or loosen the artificial chords 104.

In one implementation, echo measurements, x-rays or other monitoring are performed while the artificial chords 104 are cinched. As the chords are adjusted, the level of contact between the leaflet patch 102 and the mitral valve may be improved, thereby affecting the coaptation between the posterior leaflet 80 and anterior leaflet 82. Improving the coaptation can allow the leaflet patch device 100 to reduce or eliminate mitral regurgitation.

FIG. 9 illustrate the leaflet patch device 100 after implantation is completed, according to some embodiments. The catheters 110 (shown in FIG. 8) are disengaged from the left atrium anchors 106 and removed from mitral valve area. In one embodiment, disconnecting the catheters from the left atrium anchors 106 causes a locking flap for locking the winch position of the respective left atrium anchors to be exposed and then engaged, preventing loosening of the artificial chords 104.

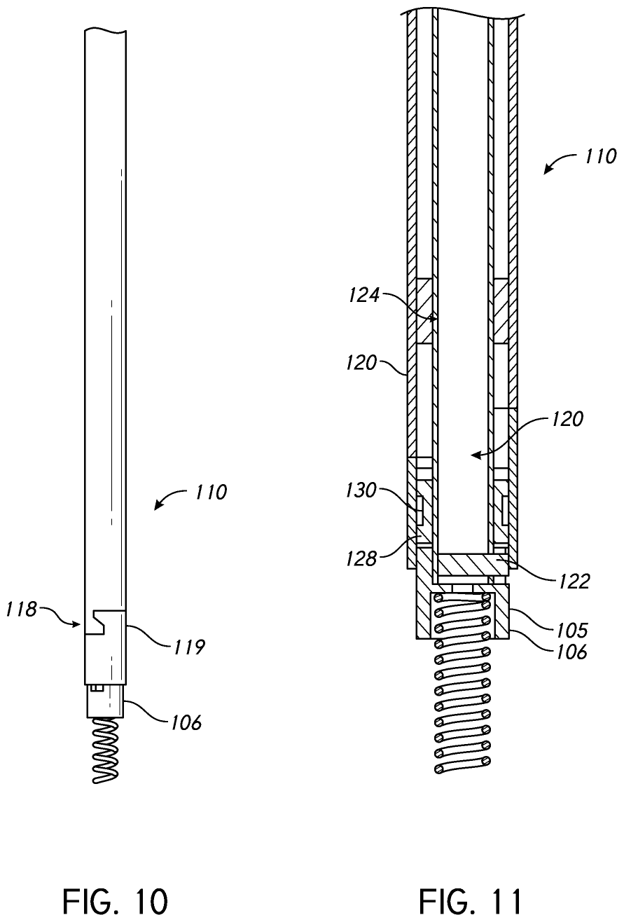

FIG. 10 illustrates the catheter 110 of FIG. 8, according to some embodiments. In some embodiments, the catheter comprises a mating end 118 configured to attach to an engagement section 119 of the left atrium anchor 106 to form a handshake connection. In one embodiment, there are complementary teeth forming the handshake connection, with one tooth formed on engagement section 119 overlapping another tooth formed on the mating end 118. As will be apparent, other shapes could be used to form the handshake connection between the mating end 118 and the engagement section 119 of the left atrium anchor.

FIG. 11 illustrates a cross-sectional view of the catheter 110 of FIG. 10, according to some embodiments. In one embodiment, the catheter 110 comprises an external sheath 120 and an inner shaft 124. The inner shaft 124 can extend past the mating end 118 of the catheter into the engagement section 119 of the left atrium anchor 106. The inner shaft 124 may extend past the engagement section 119 to the anchor base 105 of the left atrium anchor 106. The inner shaft 124 can be configured to engage with a drive formed on a top surface of the anchor base 105.

In one embodiment, the engagement section 119 comprises a winch drum 128, spool or other winching mechanism inside. The winch drum 128 may be formed between the inner shaft 124 and the exterior of the engagement section 119. A chord channel 130 can be formed circumferentially on the exterior surface of the winch drum 128. In one embodiment, the chord channel 130 holds the excess artificial chord 104 that is wound onto the winch drum.

In some embodiments, a key 122 is formed on the proximal end of the inner shaft 124, closest to the myocardium. In one embodiment, the key 122 extends perpendicularly from the external surface of the inner shaft 124. The key may be shaped as a cross, a dome, a cylinder, a cone, a prism or other shape that can catch against an engagement surface. In one embodiment, the key is configured to engage with a slot 132, a depression, or other engagement surface formed on the winch drum 128 of the left atrium anchor 106.

In one embodiment, the inner shaft 124 is independently rotatable from the catheter 110. For example, the catheter 110 may be held stationary while the inner shaft 124 is rotated to actuate the winch drum 128. Rotating the catheter 110 can drive the left atrium anchor 106 into the tissue site. In one embodiment, the inner shaft may rotate with the catheter. In another embodiment, the inner shaft remains stationary (e.g., can be held or locked) while the catheter rotates.

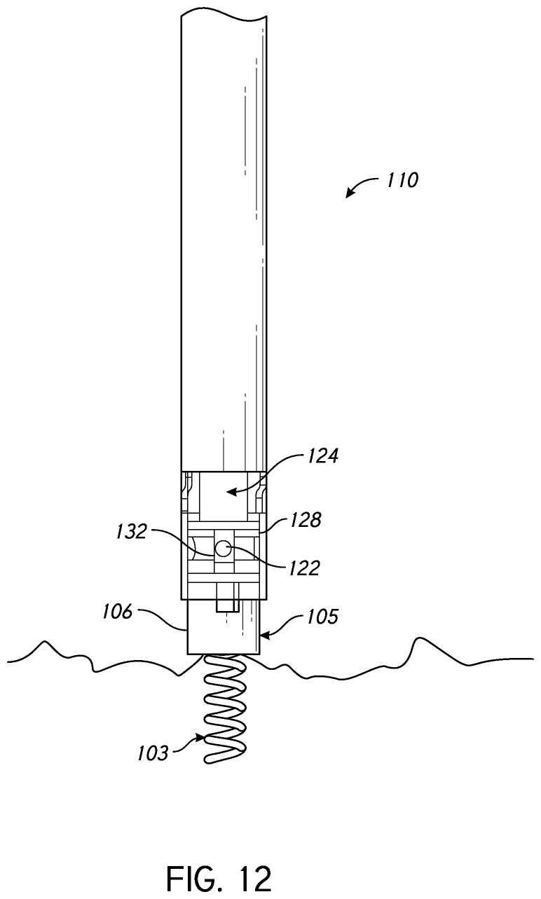

The key 122 can act as a lock when engaged with the slot 132 (shown on FIG. 12), keeping the inner shaft 124 connected to the engagement section 119 and winch drum 128. The key can engage the winch drum 128 such that rotating the inner shaft 124 can rotate the winch drum 128 to wind or unwind the artificial chord 104.

FIG. 12 illustrates the catheter 110 with an external section cut-out, according to some embodiments. The catheter 110 is shown with an external portion of the engagement section 119 cut out to show engagement of the key 122 with the winch drum 128.

In one embodiment, the slot 132 is formed lengthwise from a distal end of the winch drum towards a proximal end (close to the tissue site). The slot 132 may not extend all the way to the proximal end such that additional drum material is retained at the proximal end. The drum material may form a solid ring at the proximal end of the wind drum to better maintain the structural integrity of the winch drum.

In one embodiment, pulling back the inner shaft 124 disengages the key 122 from the slot 132 or other engagement surface of the winch drum 128. For example, a locking ring or other barrier may be formed on the distal side of the winch drum, such that the locking ring prevents the key from being pulled out of the slot unless sufficient force is applied. The catheter 110 and inner shaft can then be removed from the heart, leaving, in one embodiment, the winch drum 128 and engagement section 119 in the atrium.

Other variations of the catheter 110 are possible. In one embodiment, a first slot is formed in the anchor base 105 and a second slot formed on the winch drum 128, with the catheter 110 configured to engage the first slot to turn the anchor base 105 and engage the second slot to turn the winch drum 128. For example, the catheter may be designed to work in a first position to rotate the tissue anchor 103 attached to the anchor base 105 and rotate the winch drum 128 in a second position, where the second position is reached by pulling back on the catheter until it disengages from the first slot (in the first position) to the second slot. In one embodiment, the key 122 of the inner shaft 124 engages with the first slot in the first position and the second slot in the second position. In one embodiment, the first position is the default configuration, with the second position entered when the inner shaft 124 is pulled back from the anchor base 105. In one implementation, pulling back the inner shaft 124 disengages the key from the first slot of the anchor base 105. The key can then be engaged with the second slot of the winch drum 128.

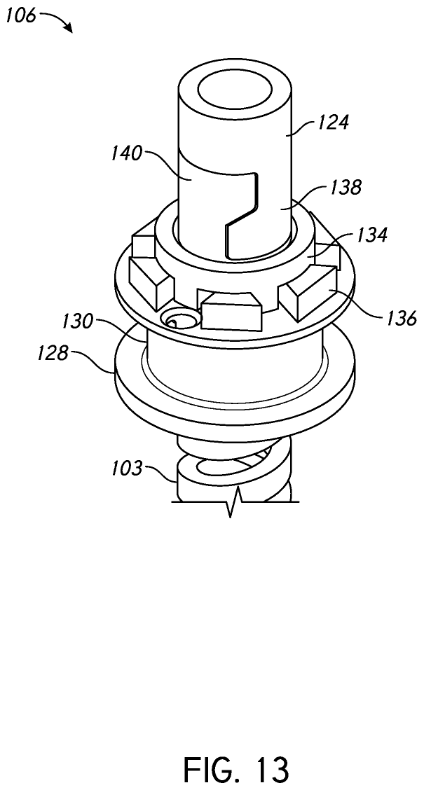

FIG. 13 illustrates a perspective view of a winch drum 128 of the left atrium anchor 106, in accordance with some embodiments. The winch drum 128 and inner shaft 124 are shown without the external sheath 120. In one embodiment, the winch drum comprises a cord channel 130, one or more engagement teeth 136, and a ratchet stopper 134.

The cord channel 130 may be formed circumferentially around the center of the winch drum, forming a spool shape with an upper disc above the cord channel and a lower disk below the cord channel The lower disc on the proximal side (to the tissue site) may be attached to the tissue anchor 103. The upper disk may have the one or more engagement teeth 136 and ratchet stopper 134 on its distal surface (away from the tissue site). The ratchet stopper 134 may be configured to stop the winch drum 128 from rotating when engaged, for example, after winding the artificial chord 104 around the cord channel 130 and reaching the desired length and/or tension.

In one embodiment, an inner shaft connector 138 at the proximal end of the inner shaft 124 forms a handshake connection with an anchor connector 140 of the left atrium anchor 106. For example, the connectors 138, 140 may form interlocking teeth. In one embodiment, a cap or pulley socket (not shown) is placed over the connectors 138, 140 to prevent disengagement. The cap may be part of the external sheath 120 and may be configured to engage with the engagement teeth 136 on the winch drum 128. In one embodiment, removal of the external sheath allows disengagement of the inner shaft 124 from the left atrium anchor 106.

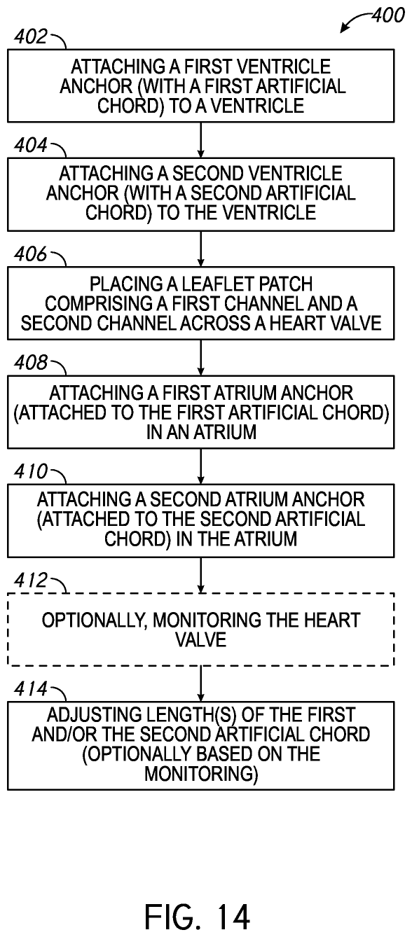

FIG. 14 provides a flow diagram representing a process for implanting a leaflet patch device 100 according to one or more embodiments disclosed herein. A health provider, such as a surgeon, can use the process during a transseptal, trans aorta, transfemoral artery, trans radial, transapical, or other surgical approach to install the leaflet patch device over a heart valve such as a mitral valve or tricuspid valve.

At block 402, the health provider attaches a first ventricle anchor to a ventricle as discussed above in FIG. 7A. The first ventricle anchor may be pre-attached to a first artificial chord or the first artificial chord may be attached to the first ventricle anchor during implantation.

At block 404, the health provider attaches a second ventricle anchor to the ventricle as discussed above in FIG. 7A. The second ventricle anchor may be pre-attached to a second artificial chord or the second artificial chord may be attached during implantation.

At block 406, the health provider places a leaflet patch comprising a first channel and a second channel across a heat valve, as discussed above in FIG. 7B. In one embodiment, the leaflet patch is partially in the ventricle and partially in the atrium. For example, the leaflet patch may be placed over a mitral valve, with the leaflet patch located partially in the left atrium and located partially in the right atrium.

At block 408, the health provider attaches a first atrium anchor in the atrium as discussed in FIG. 8. The first atrium anchor may be pre-attached to the first artificial chord or the first artificial chord may be attached during implantation.

At block 410, the health provider attaches a second atrium anchor in the atrium as discussed in FIG. 8. The second atrium anchor may be pre-attached to the second artificial chord or the second artificial chord may be attached during implantation.

At block 412, the health provider may optionally be monitoring the heart valve. For example, echo measurements, x-rays or other monitoring may be used to monitor mitral regurgitation.

At block 414, the health provider adjusts the lengths of the first artificial chord and/or the second artificial chord as discussed in FIG. 8. For example, the health provider may use a winch mechanism as discussed in FIGS. 11-12. In some scenarios, adjusting the lengths of the artificial chords are based at least in part on the optional monitoring. The health provider may tighten or loosen the chords while monitoring the effect on the mitral valve (e.g., while checking for mitral regurgitation). Once the artificial chords are tightened as desired (e.g., no additional improvements detected), the health provider can complete the implantation process.

### Additional Embodiments

Depending on the embodiment, certain acts, events, or functions of any of the processes or methods described herein can be performed in a different sequence, may be added, merged, or left out altogether. Thus, in certain embodiments, not all described acts or events are necessary for the practice of the processes. Moreover, in certain embodiments, acts or events may be performed concurrently, rather than sequentially.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

What is claimed is:

1. A device for placing a leaflet patch over a heart valve, the device comprising:
a first atrium anchor configured to be placed in an atrium of a heart;
a second atrium anchor configured to be placed in the atrium;
a first ventricle anchor configured to be placed in a ventricle of the heart;
a second ventricle anchor configured to be placed in the ventricle;
a first artificial chord configured to attach to the first atrium anchor and the first ventricle anchor;
a second artificial chord configured to attach to the second atrium anchor and the second atrium anchor; and
a leaflet patch configured to cover a leaflet of a heart valve, the leaflet patch partially in the atrium and partially in the ventricle, the leaflet patch comprising a first channel configured to contain the first artificial chord and a second channel configured to contain the second artificial chord.

2. The device of claim 1, wherein the first atrium anchor comprises an anchor base attached to a tissue anchor, the anchor base configured to rotate with a catheter and a winch drum configured to rotate with an inner shaft, and wherein rotation of the winch drum is independent from rotation of the anchor base and the rotation of the winch drum causes the first artificial chord to wind around the winch drum.

3. The device of claim 2, wherein a key of the inner shaft is configured to engage with a slot of the winch drum in a first position of the inner shaft proximal to a tissue site.

4. The device of claim 2, wherein the second atrium anchor comprises a second anchor base attached to a second tissue anchor, wherein the second anchor base is configured to rotate with a second catheter, and a second winch drum configured to rotate with a second inner shaft, and wherein rotation of the second winch drum is independent from rotation of the second anchor base and the rotation of the second winch drum causes the second artificial chord to wind around the second winch drum.

5. The device of claim 2, wherein the catheter is configured to rotatably engage with the anchor base and rotation of the catheter is configured to drive the anchor base into a tissue site.

6. The device of claim 5, wherein the inner shaft is configured to rotatably engage with a slot of the winch drum, rotation of the inner shaft is configured to rotate the winch drum, the inner shaft is located within the catheter, and the inner shaft and the catheter are independently rotatable.

7. The device of claim 6, wherein the catheter comprising the inner shaft is pre-attached to the first atrium anchor.

8. The device of claim 1, further comprising a transseptal guide sheath for inserting the device into the heart.

9. The device of claim 1, wherein the heart valve is a mitral valve and the leaflet patch is configured to cover a posterior leaflet of the mitral valve.

10. The device of claim 1, wherein the first channel is formed on a first edge of the leaflet patch and the second channel is formed on a second edge of the leaflet patch.

11. The device of claim 1, wherein the leaflet patch is substantially rectangular.

12. The device of claim 1, wherein the first artificial chord is pre-attached to the first atrium anchor and the first ventricle anchor.

13. The device of claim 12, wherein the second artificial chord is pre-attached to the second atrium anchor and the second ventricle anchor.

14. A method for treating a heart valve, said method comprising:
attaching a first ventricle anchor in a ventricle of a heart, the first ventricle anchor connected to a first artificial chord;
attaching a second ventricle anchor in the ventricle, the second ventricle anchor connected to a second artificial chord;
placing a leaflet patch across a heart valve, the leaflet patch partially in an atrium of the heart and partially in the ventricle, the leaflet patch comprising a first channel and a second channel, the first artificial chord running through first channel, the second artificial chord running through the second channel;
attaching a first atrium anchor in the atrium, the first atrium anchor connected to the first artificial chord; and
attaching a second atrium anchor in the atrium, the first atrium anchor connected to the first artificial chord.

15. The method of claim 14, further comprising adjusting the first artificial chord by using a first winching mechanism of the first atrium anchor.

16. The method of claim 15, further comprising adjusting the second artificial chord by using a second winching mechanism of the second atrium anchor.

17. The method of claim 15, further comprising monitoring mitral valve regurgitation during the adjustment of the first artificial chord, wherein the adjustment is based at least partly on the monitoring of the mitral valve regurgitation.

18. The method of claim 14, wherein attaching the first atrium anchor comprises driving the first atrium anchor into a tissue site of the heart using a catheter rotatably engaged with the first atrium anchor.

19. The method of claim 18, further comprising:
holding the catheter stationary; and
adjusting the first artificial chord by independently rotating an inner shaft within the catheter, the inner shaft rotatably engaged with a winching mechanism of the first atrium anchor.

20. A device for placing a leaflet patch over a heart valve, the device comprising:
a first artificial chord configured to attach to a first anchor in a first chamber of a heart and a second anchor in a second chamber of the heart;
a second artificial chord comprising a first end configured to attach to a third anchor in the second chamber of the heart and a second end configured to attached to the first anchor or a fourth anchor in the first chamber of the heart; and
a leaflet patch configured to cover a leaflet of a heart valve, the leaflet patch partially in the first chamber and partially in the second chamber, the leaflet patch comprising a first channel configured to contain the first artificial chord and a second channel configured to contain the second artificial chord.
